(19) United States
(12) Patent Application Publication
(10) Pub. No.: US 2022/0059211 A1
(43) Pub. Date: Feb. 24, 2022

(54) METHOD FOR SUPPORTING A USER OF A VENTILATOR, AND VENTILATION SYSTEM

(71) Applicant: Loewenstein Medical Technology S.A., Luxembourg (LU)

(72) Inventor: Matthias SCHWAIBOLD, Karlsruhe (DE)

(21) Appl. No.: 17/403,997

(22) Filed: Aug. 17, 2021

(30) Foreign Application Priority Data
Aug. 18, 2020 (DE) 102020121680.7

(51) Int. Cl. G16H 20/40; G16H 40/67; G16H 15/00; G16H 10/20; G16H 50/70; G05B 15/02; A61M 16/00; G09B 5/02

(52) U.S. Cl. CPC G16H 20/40; G16H 40/67; G16H 15/00; G16H 10/20; A61M 2205/3592; G05B 15/02; A61M 16/024; G09B 5/02; G16H 50/70

(57) ABSTRACT

A method for assisting a user of a ventilator (1), comprising the following steps:
coupling a mobile terminal (2), which comprises a control algorithm, to the ventilator (1) for the exchange of data between the terminal (2) and the ventilator (1);
transmitting data registered on the ventilator (1) to the terminal (2), wherein the data include a ventilation parameter acquired by the ventilator (1);
evaluating the transmitted data on the terminal (2), and/or by means of a processor device that communicates with the terminal (2), in order to provide at least one user information item such that the user, irrespective of their medical expertise, is provided with an evaluation and/or assistance based at least partially on the ventilation parameter;
outputting the user information item by means of the terminal (2).

### CROSS-REFERENCE TO RELATED APPLICATIONS

The present application claims priority under 35 U.S.C. § 119 of German Patent Application No. 102020121680.7, filed Aug. 18, 2020, the entire disclosure of which is expressly incorporated by reference herein.

### BACKGROUND OF THE INVENTION

### 1. Field of the Invention

The present invention relates to a method for assisting a user of a ventilator and in particular of a home ventilator. The invention furthermore relates to a ventilation system for carrying out such a method.

### 2. Discussion of Background Information

Home ventilation can sometimes offer the advantage that the patient does not need to visit a clinic and can remain at home in their familiar environment. However, this also means that the patient must operate the ventilator substantially independently. Ventilators have therefore become known which, even for users without medical or technical prior knowledge, offer a high level of operating convenience and can be used at home without the need for constant monitoring by medical personnel.

It has however been found here that, in the case of home ventilation, aside from the actual operation of the ventilator, there are often many further reasons why the user requires or desires contact with the caregiver. This relates sometimes to the monitoring of the therapy behavior and, for example, the length of time for which and the frequency with which the ventilator is used. A further aspect is that the motivation for the therapy can rapidly abate owing to the infrequent contact with the caregiver. This is however highly important for the therapeutic success. A further problem in the case of home ventilation is also that concerns and needs or even desires can be expressed only when a visit to the caregiver or physician is possible. The concerns and needs include for example the fact that falling asleep whilst wearing a respiration mask is often very difficult.

In view of the foregoing, it would be advantageous to be able to provide an improved support of a user of a ventilator and in particular, of a home ventilator. Here, the support should promote self-help and at least partially replace, or provide preparation for, contact with a caregiver.

### SUMMARY OF THE INVENTION

The present invention provides a method and a ventilation system as set forth in the independent claims. The subclaims relate to refinements and advantageous embodiments. Further advantages and features will emerge from the general description and from the description of the exemplary embodiments.

The method according to the invention serves for (in particular telematically) assisting a user of a ventilator and in particular of a home ventilator and/or sleep therapy unit. The method comprises at least the following steps (in this or some other expedient sequence). Coupling at least one mobile terminal to the ventilator (by means of in each case one transmission device) for the exchange of data between the ventilator and the terminal. The terminal comprises, in particular, at least one control algorithm. In particular, the control algorithm is installed on the terminal and is for example in the form of an application or app. Transmitting data registered on the ventilator to the terminal. The data include at least one ventilation parameter acquired by the ventilator (in particular sensorily by sensor means). Evaluating the transmitted data on the terminal and/or on the ventilator, in particular by means of a processor device of the terminal and/or by means of the control algorithm. In addition or alternatively, the evaluation of the transmitted data may be performed by means of at least one processor device which communicates with the terminal or with the ventilator, and in particular by means of a server. Such an evaluation serves for providing at least one user information item (which is in particular understandable irrespective of prior medical knowledge). Said user information item is in particular such that the user, irrespective of their medical expertise, is provided with an evaluation and/or assistance which is based at least partially on the ventilation parameter and/or an evaluation and/or assistance which at least partially takes into consideration the ventilation parameter and/or an evaluation and/or assistance which relates at least to the use of the ventilator and/or to the therapy. Outputting and preferably displaying the user information item by means of the terminal at least to the user. The outputting is performed in particular by means of an output device of the terminal. In particular, the output device comprises at least one display device, for example a display panel or the like.

In a further embodiment, the data may additionally be transmitted from the terminal to the ventilator, wherein the data are compiled locally by the terminal and/or by an auxiliary unit.

The terminal may also serve as a communication hub or modem for the ventilator. The terminal may receive data such as respiratory flow curves, respiration frequency, present therapy pressures, present leakage and the like from the ventilator and transmit these data onward.

For example, the terminal may transmit the data internally to another application, for example via an SDK/API and/or some other software interface that evaluates these data. Alternatively, the terminal may transmit the data via another interface of the terminal to an auxiliary unit. Auxiliary units may for example be a diagnostic unit or a data recorder in the surroundings of the patient. In some embodiments, the terminal may also transmit the data by remote communication (for example mobile radio or WLAN) to an auxiliary unit of a remote station, for example to a cloud software or to a physician's software.

The terminal may conversely also receive various data and transmit these to the ventilator.

Said data may for example be data compiled locally. Locally compiled data may be compiled for example by at least one auxiliary unit. Auxiliary units may for example be diagnostic units. Auxiliary units may also be thermometers or air humidity sensors or the like that provide data relating to room temperature and air humidity and the like.

Alternatively, they may also be data that the terminal receives from a remote source. Said data may for example be modified ventilation parameters which are stored by the medical caregiver and which the terminal receives from a storage location and/or from the ventilator.

The data may also be general data. General data may for example include the responsible caregiver at the patient's place of residence, this being displayed to the patient. General data may also include weather data at the place of residence of the patient, which can be utilized for an optimization of the ventilation and/or respiratory air humidification and/or respiratory air warming parameters.

By means of these data collected by the terminal and transmitted to the ventilator, the ventilator can optimize its therapy parameters, specifically for example therapy pressures and/or trigger parameters and/or parameters of the respiratory air humidifier and/or of the warming of the respiratory air.

Alternatively, the terminal may itself ascertain the optimized parameters on the basis of the data and communicate said optimized parameters to the ventilator.

When the ventilator and terminal have been connected, for example by Bluetooth, the ventilator and/or the terminal may seek further possible communication partners in parallel. For example, the terminal may comprise a second Bluetooth channel which it uses to seek further communication partners.

A further communication partner may for example be a diagnostic unit such as pulse oximeters, CO2 measuring units or polygraphs.

If a further communication partner has been found, this can connect directly to the terminal and/or to the ventilator. The connection preferably takes place directly to the ventilator.

If a further communication partner is found, the terminal may communicate to the ventilator that this communication partner exists, and may initiate an ending of the connection between terminal and ventilator. The ventilator can thus subsequently establish a connection to the further communication partner.

The method according to the invention offers numerous advantages. One considerable advantage is offered by the communication between ventilator and terminal, and the user information item thus generated on the terminal. The caregiving in the case of a home therapy is thus considerably improved and simplified. For example, the qualified personnel do not need to be visited as frequently, or do not need to be on site as frequently. It is particularly advantageous here that the assistance is understandable even for medical or technical laypersons.

With the invention, the patient can in many cases particularly conveniently help themselves. Furthermore, the patient is better involved in the therapy and has the ability to independently evaluate and improve the therapy or their behavior. In particular in the case of chronically ill patients, who often perceive the necessary monitoring by physicians as an undesired dependency, this can have a positive effect on the therapy and boost the patient's self-confidence.

A further advantage of the invention is that terminals generally have high-powered processors and high-resolution displays. These are therefore particularly suitable for the evaluation and generation of the user information item. At the same time, the processor power and display in the ventilator can be kept simpler. In this way, it is in turn possible for the lifetime of battery-operated ventilators to be considerably lengthened. It is furthermore possible for costs to be saved, and at the same time for reliable and safe ventilation to be ensured. With the terminal, the user is therefore provided with a better insight into their therapy and can communicate their concerns and needs and receive an evaluation of the therapy behavior. This would otherwise be possible only by way of a visit to or communication with a caregiver.

It is possible and advantageous that at least one stored therapeutic objective is taken into consideration for the provision of the user information item. The objective is in particular at least intermittently also stored in the terminal. In particular, the objective is created by the caregiver. Particularly targeted evaluation or assistance is thus possible.

The evaluation preferably evaluates a therapy behavior of the user. It is preferable if, for this purpose, a frequency and/or duration and/or intensity of the therapeutic use of the ventilator are evaluated. In particular, so-called compliance parameters (that is to say in particular the compliance of the patient with their therapy and its requirements) are evaluated. For this purpose, said parameters are in particular automatically acquired and registered by the ventilator.

In all embodiments, it is preferred that the evaluation comprises at least one statement relating to mask leak-tightness (leakage) and/or relating to the frequency of resting respiratory events (AHI). In particular, for this purpose, the number and/or frequency of the AHI is displayed. For the mask leak-tightness, it is the case in particular that an evaluation which qualitatively characterizes the mask leak-tightness is output (for example with a gradation from good to poor). Such an evaluation may also be implemented graphically, for example in the form of stars or smileys.

It is possible and advantageous that the evaluation comprises at least one points system and/or grading system and/or at least one evaluation system with (graphical and/or animated) symbols and/or at least one reward system. Combinations of such systems are also possible. The motivation for the therapy can thus be further increased.

It is advantageous and preferable that a record of the reward system is dependent on an achievement of a stored therapeutic objective. In particular, the reward system takes into consideration a measure for a deviation of the real therapy behavior of the user from the stored therapeutic objective. A previously defined therapy behavior may also be stored as an objective. The reward is in particular symbolic. The reward may also be coupled to other functions. For example, gaming functions may be unlocked as a reward. In particular, with the reward system, improvements in the therapy behavior are rewarded. This offers particularly illustrative feedback regarding the therapy behavior without the need for another visit to the caregiver.

At least one previous evaluation is preferably taken into consideration in the evaluation. It is also possible that at least one (voluntary) objective created or stored by the user is taken into consideration. Progress can thus be particularly easily recognized in accordance with the previous evaluation or voluntary objective.

The assistance preferably comprises at least one instruction for using the ventilator and/or the terminal and/or at least one therapy explanation (which is in particular optimized for laypersons). Preferably, the assistance is provided by way of at least one video and/or at least one animation, or comprises at least one such video and/or animation. The assistance may be implemented by way of at least one targeted connection to a video portal and/or to a video on a server.

In one advantageous embodiment, it is provided that, as assistance, at least one report is generated for a caregiver and is in particular loaded onto a defined server. It is also possible that, as assistance, consultation of a caregiver is recommended. The user can thus receive feedback and/or further assistance promptly and without an additional visit.

The report may include the user information item and/or further data. The further data serve in particular for supplementing the data provided to the user in the user information item with medical and/or technical information items for the caregiver. It is possible that the report and/or the further data are visible only to the caregiver. It is possible that the caregiver automatically receives a message regarding the created and/or stored report. The caregiver may be provided with access to the server on which the report is stored. Such a server is for example a cloud or part of a cloud. The report is in particular configured such that the caregiver is informed of the present state of affairs. For example, information is provided regarding the problems or progress identified by the ventilator, terminal and/or user.

The consultation of the caregiver may be proposed and/or automatically implemented. For example, for this purpose, a telephone call may be made or an appointment with the caregiver may be arranged. For this purpose, a calendar stored in the terminal may be taken into consideration. The placing of an emergency call is also possible. In particular, the severity of the problem on which the assistance is based is taken into consideration here.

It is possible and preferred that the caregiver can access the terminal and/or the ventilator via a data transmission connection and for example a remote process. In particular, the caregiver can interrogate further data, and/or adjust settings, therein. It is also possible that the caregiver can send messages and/or appointment arrangements to the terminal. It is thus possible, by way of the terminal, to initially automatically ascertain the nature of concerns or problems. Should contact with a caregiver then nevertheless be necessary, said caregiver can then assist further, or ask targeted follow-up questions.

In a particularly preferred and advantageous embodiment, at least one function for a digital diary is stored on the terminal. In particular, the user can use the diary to log their experiences with the therapy. In particular, the diary is taken into consideration for preparing the user information item and in particular the assistance and/or for the report and/or for the evaluation.

In all embodiments, it is particularly preferred that at least one input mask is displayed on the terminal. The user can input subjective and/or objective additional information items into the input mask. In particular, the provision of the user information item is performed at least partially in a manner dependent on the additional information item. In particular, for the provision of the user information item, the additional information items are set off against the data provided by the ventilator, and in particular the ventilation parameter. For the provision of the user information item, the additional information items and the data provided by the ventilator may be taken into consideration in weighted form. The user information item is in particular calculated in a manner dependent on the additional information item and also on the data provided by the ventilator. Preferably, the user information item is calculated in accordance with a validated logic, which is in particular part of the control algorithm, from the additional information items and the data provided by the ventilator.

It is preferred and advantageous that the additional information items are interrogated in a time-dependent manner and/or on the basis of a control command of the ventilator and/or on the basis of a request by the user. A time-dependent interrogation is performed in particular at certain intervals and/or upon certain events and for example upon switching-on of the ventilator. A control command that initiates such an interrogation may be issued for example as a result of an operating error and/or as a result of a fault and/or as a result of an identified particular respiratory event. For example, additional information items may be interrogated in the event of interruptions in respiration, leakages at the mask and/or less frequent use thereof, or the like being detected. An interrogation based on a request by the user may be performed for example if said user presses a help button.

The subjective additional information items are in particular ones which are not acquired, and/or are not acquirable, by sensor means. In particular, the additional information items include an occurrence of pressure points and/or fitting difficulties at respiration interfaces. The subjective additional information items are taken in particular from a group of additional information items at least comprising: dissatisfaction with the therapy, difficulties with the unit, handling problems, perceived side effects, perceived pressure points, noise perception, health perception, sleeping habits. In addition or alternatively, the group of subjective additional information items may at least also comprise: respiratory distress in general, respiratory distress in specific situations such as lying, sitting, walking, eating, walking up or down stairs, running, in the morning, in the evening, at night, after switching off the ventilator etc.; secretion formation in the nose; secretion formation in the throat; secretion formation in the upper respiratory tracts; cough competence; quality of life in general, disease-related quality of life, daytime fatigue, daytime drowsiness. In this regard, it is also possible for a value to be indicated on a scale for satisfaction.

The objective additional information items are taken in particular from a group of additional information items which comprise at least: resting pulse rate, active pulse rate, age, weight, body height, body mass index (BMI), blood pressure values, pre-existing medical conditions, activity, movement, sleep behavior (sleep activity). In addition or alternatively, the group of objective additional information items may at least also comprise: lung function, in particular expiratory peak flow, oxygen saturation of the blood, CO2 content of the blood, CO2 content of the exhaled air, organic composition of the exhaled air (in particular volatile organic molecules), stroke volume of the heart, minute volume of the heart, heart frequency, body temperature, ambient conditions (temperature, air pressure, air contamination), color of the sputum, composition of the sputum, sleep quality, sleep stages, sleep fragmentation, sleep/waking information. The objective additional information items are in particular acquired by sensor means and/or are acquirable by sensor means. Such parameters acquired by sensor means may be manually input and/or automatically read in (into the app), for example by way of a Bluetooth connection, a coupling of the app on the terminal, for example by way of an SDK, or a reading-in of the sensor data from a cloud server by API.

It is possible that the objective additional information items are provided at least partially by the terminal and/or the ventilator. In particular, sensor means and/or health applications of the terminal can be accessed for this purpose. It is also possible and preferable that the objective additional information items are health parameters measured by the user themself, for example blood pressure values.

The input mask preferably comprises at least one questionnaire with a multiplicity of stored questions. In particular, the questions are selected interactively and/or dynamically.

In exemplary embodiments of the input mask and in particular of the questionnaire, the following steps are performed in their entirety, or at least in part:
In the case of insufficient use (preferably less than 6 hours per day, particularly preferably less than 4 hours per day or optionally also less than 2 hours per day) or a decrease in use (preferably by more than 1 hour, particularly preferably by more than 2 hours per day), questions are targetedly asked regarding a lack of motivation, and/or an explanation regarding the purpose of the therapy is offered, and/or questions are asked regarding possible handling problems during the therapy and/or regarding possible side effects of the therapy and/or regarding a possible infection of the upper respiratory tracts and/or increased secretion formation, which make respiration with a respiration mask more difficult.
In the case of increasing or increased mask leakage (preferably median leakage of over 20 l/min or over 15 l/min or 95^{th} percentile of the leakage over 50 l/min or high leakage over a proportion of more than 10% of the therapy time), questions are targetedly asked regarding possible leakage-induced side effects. Here, it is particularly preferably the case that the threshold value of the leakage that triggers the targeted questions is selected to be more sensitive if lower or falling usage time per day is measured.
In the case of an increased respiratory frequency (preferably over 20 bpm or over 25 bpm) or in the case of an increasing respiratory frequency (preferably by over 3 bpm in relation to a comparison value from a chronologically preceding comparison period), questions are asked regarding increased or elevated respiratory distress.

The input mask, in particular the questionnaire, is preferably created and/or adapted in a manner dependent at least on the ventilation parameter acquired by the ventilator and/or on a previously provided user information item and/or on at least one user input. The input mask may also be created and/or adapted on the basis of a control command of the ventilator and/or on the basis of other data provided by the ventilator. Such a user input may for example be an actuation of a help button and/or a particular menu selection on the terminal. Such buttons are in particular arranged on an operator control device of the ventilator. The input mask may be generated from stored individual elements and/or selected from a multiplicity of stored input masks.

In particular, defined (in the control algorithm) ventilation parameters are taken into consideration for the provision of the user information item. Here, it is possible and preferred that those defined ventilation parameters which are not available for the evaluation and/or which have not been or cannot be provided by the ventilator are substituted and/or supplemented by (subjective and/or objective) additional information items. In particular, the additional information items required for this purpose are purposely interrogated by way of the input mask. The input mask is in particular correspondingly adapted for this purpose. This allows a considerable improvement in the evaluation and assistance. In particular, the control algorithm has stored in it which additional information items are necessary for substitution or supplementation.

Preferably, the ventilation parameters used for the provision of the user information item are selected at least partially in a manner dependent on the additional information items.

In particular, the provision of the user information item is performed interactively. In particular, the user information item is graphically depicted for this purpose. In particular, for this purpose the inputting of the subjective additional information items and/or further user inputs is possible while the user information item is being depicted. In particular, the user information item is newly created and/or dynamically adapted by way of such inputs.

In all embodiments, it is particularly preferred that the user information item is created in a time-dependent manner and/or on the basis of a control command of the ventilator and/or on the basis of a request by the user. Such a creation of the user information item is in particular at least partially configured as has been described above for the interrogation of the additional information items.

In particular, a time period of a multiplicity of therapy hours and/or of a multiplicity of therapy days is taken into consideration for the provision of the user information item. Longer or shorter time periods are also possible.

In one particularly preferred and advantageous embodiment, it is possible that the provision of the user information item is performed during an ongoing therapy. This allows particularly fast and prompt feedback and/or assistance. A further advantage is that it is much more convenient during the therapy to use the mobile terminal than, for example, to select a menu on the ventilator that is presently performing the therapy.

In particular, during an ongoing therapy, data are transmitted continuously from the ventilator to the terminal. In particular, the user information item is thus continuously updated. “Continuously” is to be understood in particular to mean both a constant repetition and a repetition at particular points in time or an irregular repetition.

In a particularly preferred and advantageous embodiment, the user information item is updated upon a respiratory event and preferably upon every new respiratory event. Such a respiratory event comprises in particular an inhalation and/or an exhalation. Other respiratory events, for example particularly fast or slow or shallow respiration or interruptions in respiration or the like, are also possible. The user is thus provided with prompt feedback, for example in real time or virtually in real time, regarding their respiration. In this way, the respiration assistance provided by the ventilator can be supplemented in targeted fashion by the cooperation of the user. This is particularly advantageous for example if respiration without a ventilator is provided as a therapeutic objective.

In a particularly advantageous and preferred refinement, it is provided that the user information item is at least partially projected by means of at least one projection device of the terminal into the surroundings, in particular such that the user information item can be at least partially visually perceived even without directly viewing the terminal. In particular, the terminal comprises at least one projection device. In particular, the terminal comprises at least one display device and in particular a display panel. In particular, the projection device is provided by the display device or comprises at least one such display device. Also possible is a projection device which is formed at least partially separately from the display device. In particular, the user information item is projected such that it is not necessary to look at the terminal, and it is in particular not necessary to look at the display devices thereof, in order to perceive the user information item.

It is however also possible for the user information item to be at least partially depicted on a display device of the terminal. It is then for example possible for the user to hold the terminal in their hands and lie comfortably in bed and follow the user information item on their smartphone without having to direct their view to the ventilator.

It is also possible and preferred that the user information item is output at least partially acoustically and/or haptically. For example, for this purpose, a rendition of tones and/or sounds and/or melodies and/or other types of acoustically perceptible sequences is performed. The haptic rendition may be performed for example by way of a vibration or the like. This, too, allows the user information item to be perceived without the need to directly view the terminal.

It is preferred if, by means of the projection device, different lighting effects are projected into the surroundings. It is also possible that, by means of the projection device, visual structures are generated on at least one projection surface in the surroundings. The projection surface is for example a wall and/or a ceiling of a room. For this purpose, it is for example possible for different colors and/or patterns to be provided in each case for inhalation and exhalation. It is also possible that different acoustic and/or haptic signals are used in each case for inhalation and exhalation. The user can thus be provided with direct feedback as regards how long they should inhale and exhale for in order to be able to achieve a good result with the therapy. It is also possible that, in this way, it is displayed when the respiration is too shallow and/or being performed too slowly.

In a likewise preferred and advantageous refinement, it is provided that the user information item indicates at least one measure for the degree to which an actual value of a ventilation parameter (presently) acquired (by sensor means) by the ventilator for the respiration of the user differs from a setpoint value of the ventilation parameter that is stored (in the ventilator). As ventilation parameter, it is for example possible for the respiratory frequency or other suitable ventilation parameters to be provided. The measure may be depicted for example by way of corresponding colors or tones or the like. It is for example possible for green light to be projected if the respiration corresponds particularly closely to the setpoint values, and for red light to be projected if the respiration deviates from the setpoint value to too great a degree.

Also possible is a user information item which is configured as an animation or comprises moving graphical elements. It is then possible, for example, for the user to view their smartphone and, with the aid of the animation, follow whether their respiration corresponds to the specification. In the animation, the measure may be illustrated for example by the position or color or size of objects. This is for example also highly advantageous in the case of children as patients.

It is preferred and particularly advantageous if the measure is configured as at least one animation which can be influenced interactively by the respiration process of the user, such that the user is, from the change in the animation, provided with direct feedback as regards said respiration process. In particular, the user is provided with direct feedback as regards how close their respiration is to the respiration required for a therapy. Such an animation may comprise visual and/or acoustic and/or haptic elements. The animation may also be provided in the form of a computer game or of a game. The user can thus perform therapy on their respiration in playful fashion and identify, in uncomplicated fashion, whether and in what direction they should adapt their respiration (gamification). For example, by means of the respiration, it must be sought to steer a virtual object through an animated environment. The movement is then for example coupled to the inhalation and exhalation.

Preferably, the method is configured as a relaxation or sleep-inducing aid during use of the ventilator and/or as a learning aid for the use of the ventilator. In particular, here, at least one stored and/or settable respiration pattern is specified by the ventilator. In particular, at least one setpoint ventilation parameter required for the respiration pattern is set. In particular, the specified respiration pattern is output by way of the user information item by means of the terminal. In particular, at least one actual ventilation parameter of the user is acquired by sensor means (by the ventilator). Preferably, here, the respiration pattern is (continuously) adapted in a manner dependent on the degree to which the actual ventilation parameter differs from the setpoint ventilation parameter. In particular, here, it is also the case that the specified respiration pattern is partially adapted to the real respiration pattern of the patient to a defined extent in order to make it easier for the user to synchronize their respiration with the specified respiration pattern. The user can thus, by observing the user information item, monitor whether their respiration corresponds to the respiration pattern whilst the respiration pattern is simultaneously also adapted (slightly) to the user's respiration pattern. An assimilation of the specified respiration pattern with the real respiration pattern of the user takes place in particular at least once per breath.

The applicant reserves the right to separately claim such a method as a relaxation or sleep-inducing aid during use of the ventilator and/or as a learning aid for the use of the ventilator. This method according to the invention also achieves the above-stated object in a particularly advantageous manner. Such a method is preferably at least partially configured as has been described above for the other method according to the invention. It is possible and preferred that the features of both methods can be expediently combined with one another and/or exchanged for one another. In particular, the method serves not for the therapy itself but only for the preparation for or facilitation of an intended respiration therapy. The respiration pattern or the specified respiration is preferably settable and/or selectable from a group of respiration patterns. The user can thus, depending on the exercise or habits with regard to falling asleep or therapy requirements, adapt the exercise according to their desires.

The respiration pattern particularly preferably defines when and/or for how long inhalation should be performed. In addition or alternatively, the respiration pattern may define when and/or for how long exhalation should be performed. In particular, for this purpose, in each case one visual and/or acoustic and/or haptic signal for the inhalation and/or exhalation is output in time-controlled fashion, as a user information item, by way of the terminal. For this purpose, the user information item is in particular configured as described above. It is particularly preferable if the projection device and/or the display device is used for this purpose.

For example, the respiration pattern may be depicted by way of color changes and/or animations. In addition or alternatively, the respiration pattern may be depicted by way of tones or sounds and, for example, the sound of the sea or birdsong or melodies. It is particularly preferably possible for the respiration pattern to be influenced interactively by the user, for example as has been described above for the interactive animation. It is thus possible to learn correct respiration, or how to fall asleep quickly, in playful fashion (gamification). For example, it is possible here, too, for a virtual object to be steered through an animated environment by way of the respiration, which should correspond to the respiration pattern.

The ventilation system according to the invention comprises at least one ventilator and at least one mobile terminal. The ventilator is suitable and configured for being operated in accordance with at least one method according to the invention as described above. In particular, the ventilation system is suitable and configured for carrying out at least one of the methods according to the invention. The ventilation system according to the invention also achieves the above-stated object in a particularly advantageous manner.

In particular, the method serves for assistance in respiration therapy that is or can be performed by means of the ventilator. In particular, the assistance is performed before and/or after and/or during use.

The data and in particular the ventilation parameter may be transmitted (directly) in the form of raw data. The data and in particular the ventilation parameter may undergo processing, for example a conversion and/or selection or the like, before being transmitted. In particular, the coupling comprises a security system. For example, the terminal and the ventilator must be in physical proximity at least once. In particular, a renewed coupling takes place at least partially automatically, for example upon registration in the same (WLAN) network. The transmission of data registered on the ventilator to the terminal takes place in particular via a secure connection.

The data and in particular the ventilation parameter are in particular acquired by at least one sensor means of the ventilator. The ventilation parameter is taken in particular from a group of ventilation parameters comprising at least: pressure; flow (flow rate or volume flow); respiratory frequency; tidal volume; ascertained respiratory disruptions; ventilation parameters relating to device functions and for example set therapy modes such as CPAP, APAP; acquisition of times of use (when, for how long); faults; identified operating errors.

The user information item may comprise at least one handling instruction or may be configured as such. The user information item may also comprise an action or be configured as such. For example, the automatic placing of an emergency call, and/or an establishment of contact with the caregiver, may be performed as an action. Other suitable actions, and for example communication with another processor device and/or with the ventilator, are also possible. Other visual and/or acoustic and/or haptic actions are also possible. In all embodiments, it is particularly preferred that the user information item is produced at least in graphical form and, for example, animated. In particular, the user information item comprises graphical parts and/or text parts.

It is possible that the provision of the user information item is performed at least partially in a manner dependent on unit settings and/or therapy settings of the ventilator. In particular, the therapy of the ventilator may be performed independently of the assistance and in particular of the provision of the user information item. In particular, the outputting of the user information item is performed independently of and/or in addition to an actuation of a ventilation device of the ventilator. A provision of the user information item is to be understood in particular to mean a generation of such.

The mobile terminal is in particular a smartphone or a tablet or some other type of hand-held or mobile smart device. Such terminals are for example handhelds (such as smartphones, foldable smartphones or tablets or touchpads) or other types of smart devices (for example a smartwatch) or notebooks, foldable notebooks or the like.

In the context of the present invention, the user is to be understood in particular to mean the person receiving the therapy. The caregiver is in particular a member of qualified medical and/or technical personnel or is a physician. A caregiver may for example be a member of technical support and/or a member of medical support. (Home) ventilation is to be understood in particular to mean (all) forms of therapy that use positive pressure therapy. This is in particular to be understood as including at least the following: sleep therapy (CPAP, bilevel, ASV), high-flow therapy, oxygen therapy, non-invasive ventilation, invasive ventilation, mouthpiece ventilation.

In all configurations, it is preferred that the ventilator is at least partially remote-controllable by means of the terminal. In particular, it is provided that the ventilator for performing a therapy can be started and/or stopped and/or paused and/or set by means of the terminal.

### BRIEF DESCRIPTION OF THE DRAWINGS

Further advantages and features of the present invention will emerge from the description of the exemplary embodiments, which will be discussed below with reference to the appended figures.

In the figures:

FIG. 1 is a purely schematic illustration of a ventilation system according to the invention in a perspective view;

FIG. 2 is a purely schematic block circuit diagram of the invention; and

FIGS. 3-6 show screenshots of a terminal.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

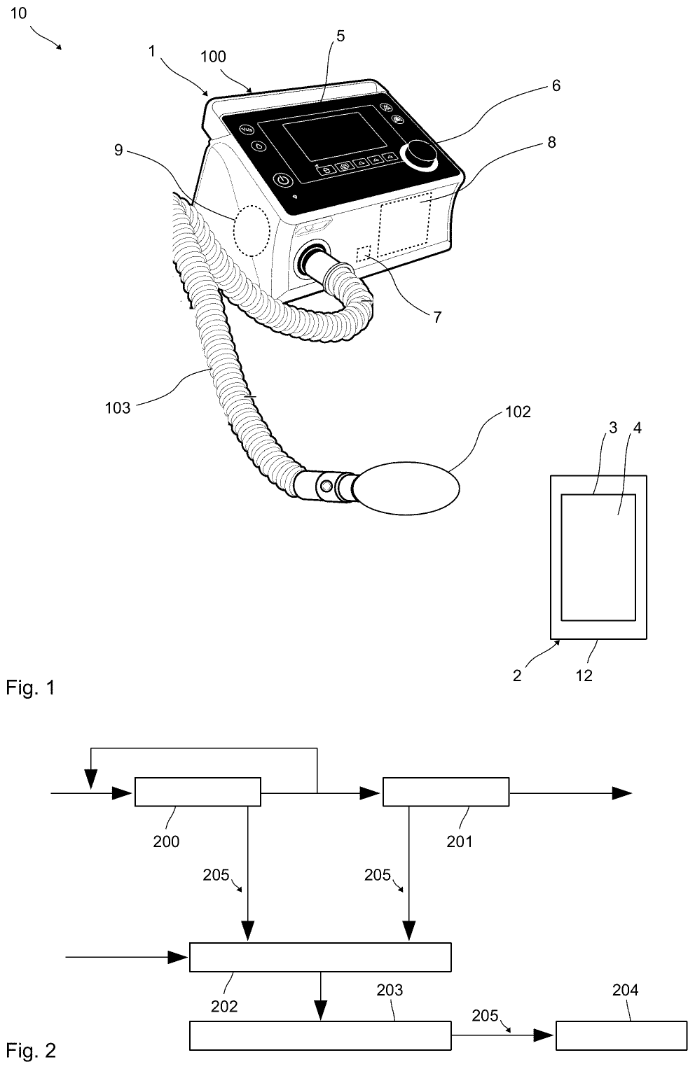

FIG. 1 shows a ventilation system 10 according to the invention with a ventilator 1, which is configured as a home ventilator 100, and with a terminal 2. The terminal 2 is configured in this case as a smartphone 12 and comprises a display device 4 with a display panel. The terminal 2 can be coupled to the ventilator 1, for example by WLAN, Bluetooth or some other form of wireless or wired transmission technology. Data can be transmitted between the coupled units 1, 2 of the ventilation system 10. The ventilation system 10 shown here is operated in accordance with the method according to the invention.

The ventilator 1 has, in the interior of its housing, a ventilation device 8 which is equipped with a fan device 9 for generating a respiratory gas flow. The respiratory gas flow is fed via a hose device 103, which is coupled to the ventilation device 8 and which has a respiration interface 102 (for example respiration mask), to the user.

Here, the ventilator 1 comprises a display device 5 and an operator control device 6. Here, it is also possible for combinations of operator control device 6 and display device 5 to be provided, for example in the form of a touch-sensitive display surface or a touchscreen. Here, the operator control device 6 comprises a help button which can be pressed in order to request help in the event of problems. It is also possible here for the ventilator 1 to be partially remote-controlled from the terminal 2.

The ventilation device 8 is in this case operatively connected to a sensor means 7 which has one or more sensors for acquiring ventilation parameters and possibly further values that are characteristic of the ventilation. For example, the sensor means 7 comprises a pressure sensor (not shown in any more detail here) which acquires the pressure conditions of the respiratory gas flow. The sensor means 7 is in this case operatively connected to a control device such that the acquired ventilation parameters can be registered, possibly evaluated, and transmitted to the terminal 2. As ventilation parameters of said type, it is also possible here for unit parameters and for example a state of charge of a battery or an error notification or the like to be transmitted.

Here, it is for example possible for CPAP ventilation or APAP ventilation to be performed by means of the ventilation device 8. For ventilation, the ventilation device 8 is for example set to a defined respiratory gas flow and/or a respiratory gas pressure. The ventilation device 8 can provide a required minimum pressure and/or compensate pressure fluctuations which are caused by the respiration activity of the user. For example, the ventilation device 8 acquires the present pressure in the respiration mask 102 by the sensor means 4 and accordingly readjusts the power of the fan device 8 until a desired ventilation pressure prevails.

The terminal 2 coupled to the ventilator 1 serves here as a communication interface between the user and the ventilator 1 and between the user and a caregiver or physician. The user can thus be telematically assisted. For this purpose, a control algorithm, in this case an application or app, is installed on the terminal. The data registered on the ventilator 1 are transmitted to the terminal 2. The transmitted data are then evaluated on the terminal 2 in order to provide a user information item. By means of the user information item, the user, irrespective of their medical expertise, is provided with an evaluation and assistance which take into consideration the transmitted ventilation parameters. The user information item is then displayed on the display device 4 of the terminal 2.

The data of the unit 1 can be interpreted by means of the terminal 2 with the app, and the user or patient is provided with feedback relating to their therapy in accordance with a validated logic. Smartphones normally have high-powered processors and good displays for such an evaluation and are particularly suitable for an interactive presentation of the evaluation. The processor power and the display in the ventilator 1 can then be kept simpler, which saves costs and nevertheless assists in ensuring good and safe ventilation.

In certain situations (certain points in time, if it is suspected that problems exist, if the patient clicks on the “problem button”), it is additionally possible here to offer a questionnaire to the patient in the app, which the patient should fill out. The statements made by the patient, for example with regard to handling problems or side effects, are combined by the app with the unit data in order to provide even more targeted feedback. This may also include the recommendation that the user report to the caregiver or caregiving physician. In this case, the app may also generate a report which relates to the acquired data and which the patient can provide to the physician.

Here, the patient can load or input health data from the terminal 2 into the app as additional information items. These may include resting pulse rate and active pulse rate, age, weight, pre-existing medical conditions, activity, movement, sleep features etc. These additional information items are optionally additionally set off against the data of the ventilator 1 in order to improve the quality of the interpretation. It is furthermore also possible for subjective additional information items to be input and taken into consideration, for example satisfaction.

With the ventilation system 10, it is also possible here to implement a method which is configured as a relaxation or sleep-inducing aid during use of the ventilator 2 and/or as a learning aid for the use of the ventilator 2. In this way, the user is provided with a relaxation/sleep-inducing aid by way of biofeedback in the app. Here, a respiration pattern is visualized as a user information item.

For this purpose, the terminal 2 specifies for the user, by way of the app, for example a relaxing respiration pattern, for example a respiratory frequency of 10 bpm with ⅓ inhalation time and ⅔ exhalation time. This pattern is however not reproduced rigidly, because the real respiration pattern and the specified respiration pattern would otherwise progressively diverge from one another. Instead, the specification is synchronized with the real respiration pattern at least once per breath, such that the patient, with their present respiration, is “collected” and in each case “pushed” a little in the direction of ideal respiration.

Here, the specification of the real respiration pattern is performed by means of a projection device 3, which is provided for example by the display device 4. For example, for this purpose, the brightness of the display is increased. The respiration pattern is projected by way of coloring of the display in different colors for inhalation and exhalation. Thus, when the terminal 2 is in a horizontal position, a pattern that specifies the real respiration pattern is generated on the ceiling or wall of the room. The exercise can thus be performed for example whilst lying down, without the need to look at the terminal 2. Alternatively or in addition, the specification of the real respiration pattern may also be performed by means of a graphical animation on the display of the terminal 2 and/or by means of an acoustic animation.

FIG. 2 shows an exemplary and in this case highly simplified block circuit diagram of certain elements of the app. Here, as user information items, a day feedback 200 is created and output daily, and a week report 201 is created and output weekly (for example on Sundays). Here, it is for example possible for personal weekly objectives to be taken into consideration and possibly rewarded. In the context of the day feedback 200, it is for example also possible for a therapy diary to be provided, with a daily facility for assigning an emoji together with a short text.

As a possible evaluation rule for the day feedback 200, it is for example possible for each therapy day to be individually classified in accordance with particular criteria into four or more categories, for example from excellent to poor. The feedback message for the individual day can then be set on the basis of the category. The individual days and their categories are then mathematically analyzed in order to provide data for the week report 201.

Feedback messages can be provided for example with regard to compliance (unit use), mask leak-tightness (leakage) and number of resting respiratory events (AHI). It is also possible for feedback provided by the user, for example their star ratings, to be taken into consideration in the day feedback 200 and/or in the week report 201.

Here, at certain intervals and for example every seven, 28 or 84 days, a therapy check 202 is started which, for this purpose, comprises an input mask with a questionnaire. The therapy check 202 may also be started on the basis of a trigger 205, for example if a help button is pressed. The occurrence of certain parameters (acquired by sensor means) or certain critical subjective or objective additional information items may also be taken into consideration as a trigger 205. A therapy check questionnaire of said type, and/or contacting of the physician, may also be recommended in the event of problems being identified. For this purpose, in particular, threshold values are stored, such that the overshooting thereof can trigger such a recommendation.

The therapy check 202 may be analyzed automatically or on the basis of a request in order to obtain an evaluation 203 of the therapy check 202. The evaluation 203 may include rewards, recommendations (for example consultation of the caregiver) and/or assistance (troubleshooting).

The evaluation 203 of the therapy check 202 is then transmitted on its own, or together with a status report and/or unit data, for example, to the caregiver or physician.

Furthermore, the transmission of the evaluation 203 may also be initiated by way of a suitable trigger 205.

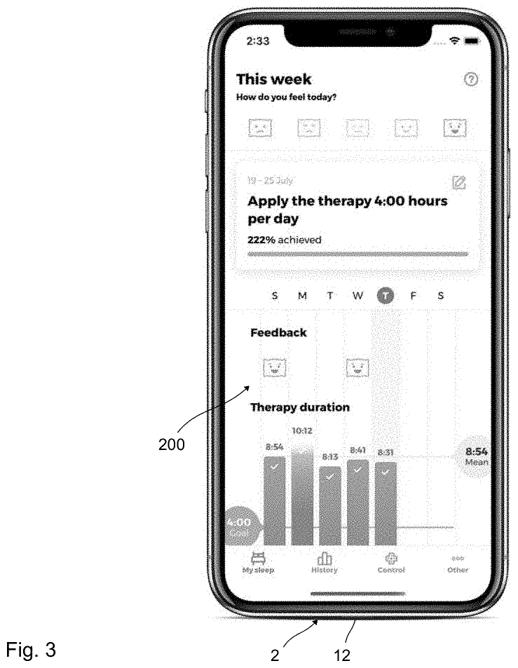

FIGS. 3 to 6 show screenshots of a terminal 2 which is configured as a smartphone 12 and which is equipped with the app.

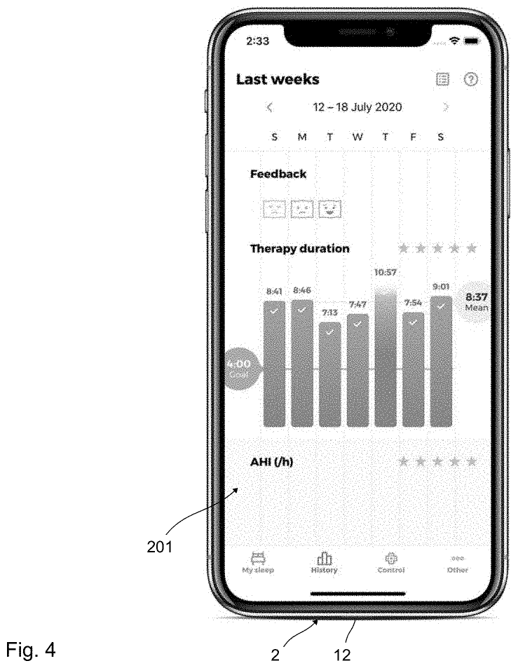

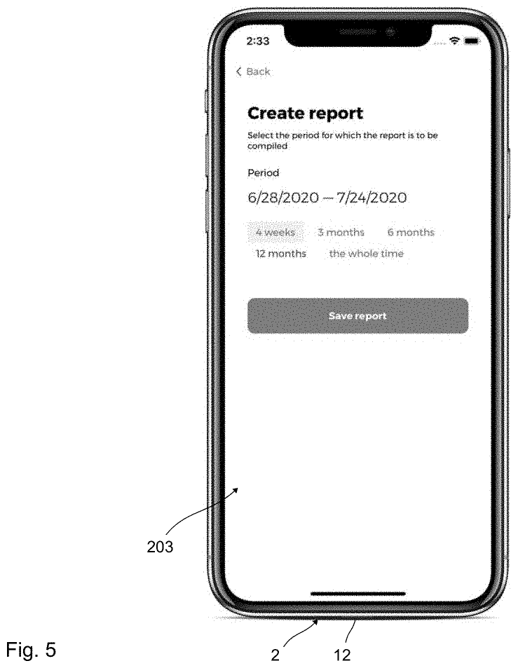

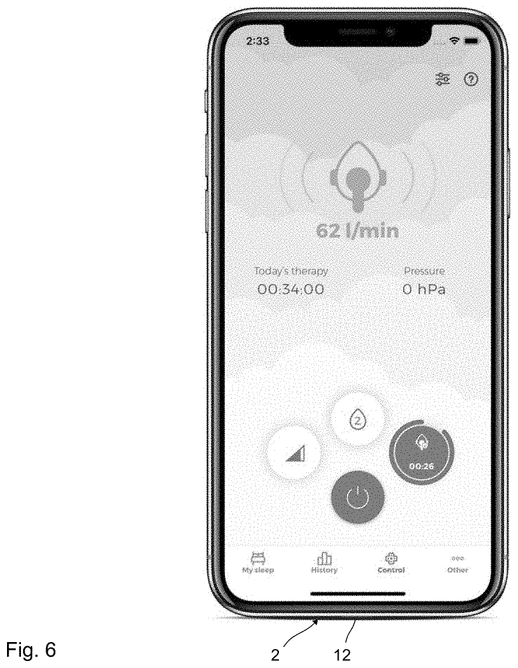

FIG. 3 shows the provision of user information items within a therapy week with, for example, instances of day feedback 200. FIG. 4 shows a week report 201. FIG. 5 shows the screen for a selection for starting an evaluation 203. FIG. 6 shows the screen when the terminal 2 is used as a remote controller for the ventilator 1.

### LIST OF REFERENCE DESIGNATIONS

1 Ventilator

2 Terminal

3 Projection device

4 Display device

5 Display device

6 Operator control device

7 Sensor means

8 Ventilation device

9 Fan device

10 Ventilation system

12 Smartphone

20 Auxiliary unit

100 Home ventilator

102 Respiration interface

103 Hose device

200 Day feedback

201 Week report

202 Therapy check

203 Evaluation

204 Transmission

205 Trigger

1-25. (canceled)

26. A method for assisting a user of a ventilator, wherein the method comprises:
coupling at least one mobile terminal, which comprises at least one control algorithm, to the ventilator for data exchange between the ventilator and the terminal;
transmitting data registered on the ventilator to the terminal, the registered data comprising at least one ventilation parameter acquired by the ventilator;
evaluating the transmitted data on the terminal and/or on the ventilator, and/or by means of a processor device that communicates with the terminal or with the ventilator, to provide at least one user information item such that the user, irrespective of its medical expertise, is provided with an evaluation and/or assistance based at least partially on a ventilation parameter;
outputting the user information item by means of the terminal.

27. The method of claim 26, wherein data are additionally transmitted from the terminal to the ventilator, the data being compiled locally by the terminal and/or by an auxiliary unit.

28. The method of claim 26, wherein for providing the user information item at least one stored therapeutic objective is taken into consideration.

29. The method of claim 26, wherein the evaluation evaluates a therapy behavior and wherein, for this purpose, a frequency and/or duration and/or intensity of a therapeutic use of the ventilator are evaluated and/or wherein the evaluation comprises at least one statement relating to mask leak-tightness and/or relating to a frequency of resting respiratory events.

30. The method of claim 26, wherein the evaluation comprises a points system and/or a grading system and/or a system with graphical symbols and/or a reward system, and wherein at least one previous evaluation and/or at least one objective created by the user is taken into consideration in the evaluation.

31. The method of claim 26, wherein the assistance comprises at least one instruction for using the ventilator and/or at least one therapy explanation, and wherein, as assistance, at least one report is generated for a caregiver and/or consultation of a caregiver is recommended.

32. The method of claim 26, wherein, on the terminal, there is stored at least one function for a digital diary, by which the user can log its experiences with a therapy, and wherein the diary is taken into consideration for preparing the assistance.

33. The method of claim 26, wherein, on the terminal, there is displayed at least one input mask into which the user can input subjective and/or objective additional information items, and wherein the provision of the user information item is performed at least partially in a manner dependent on the additional information items.

34. The method of claim 33, wherein the additional information items are interrogated in a time-dependent manner and/or on the basis of a control command of the ventilator and/or on the basis of a request by the user.

35. The method of claim 33, wherein the subjective additional information items are not acquired, and/or are not acquirable, by sensors, and comprise an occurrence of pressure points and/or leakages at respiration interfaces, and/or wherein the objective additional information items are taken from a group of additional information items at least comprising: resting pulse rate, active pulse rate, age, weight, body height, body mass index (BMI), blood pressure values, pre-existing medical conditions, activity, movement, sleep features.

36. The method of claim 33, wherein the objective additional information items are provided at least partially by the terminal and wherein sensors and/or health applications of the terminal can be accessed for this purpose, and/or wherein the objective additional information items are health parameters measured by the user itself.

37. The method of claim 33, wherein the input mask comprises at least one questionnaire with a multiplicity of stored questions.

38. The method of claim 33, wherein the input mask is created and/or adapted in a manner dependent at least on a ventilation parameter acquired by the ventilator and/or on a previously provided user information item and/or on at least one user input.

39. The method of claim 33, wherein defined ventilation parameters are taken into consideration for providing the user information item, and wherein those defined ventilation parameters which are not available for the evaluation are substituted and/or supplemented by additional information items, and wherein the additional information items required for this purpose are purposely interrogated by way of the input mask.

40. The method of claim 33, wherein ventilation parameters used for the provision of the user information item are selected at least partially in a manner dependent on the additional information items.

41. The method of claim 33, wherein provision of the user information item is performed interactively, and wherein the user information item is graphically depicted for this purpose, and wherein the inputting of the subjective additional information items and/or further user inputs is possible while the user information item is being depicted, and wherein the user information item is dynamically adapted by way of such inputs.

42. The method of claim 26, wherein the user information item is created in a time-dependent manner and/or on the basis of a control command of the ventilator and/or on the basis of a request by the user, wherein a time period of a multiplicity of therapy hours and/or of a multiplicity of therapy days is taken into consideration for providing the user information item.

43. The method of claim 26, wherein provision of the user information item is performed during an ongoing therapy, wherein, during the ongoing therapy, data are transmitted continuously from the ventilator to the terminal and the user information item is thus continuously updated, wherein the user information item is updated upon a respiratory event.

44. The method of claim 26, wherein the user information item is projected by at least one projection device of the terminal into the surroundings, such that the user information item can be visually perceived even without directly viewing the terminal, and/or wherein the user information item is implemented acoustically and/or haptically.

45. The method of claim 26, wherein the method is configured as a relaxation or sleep-inducing aid during use of the ventilator and/or as a learning aid for the use of the ventilator, and wherein at least one stored and/or settable respiration pattern is specified by the ventilator, and wherein at least one setpoint ventilation parameter required for the respiration pattern is set, and wherein the specified respiration pattern is output by the terminal (2), and wherein at least one actual ventilation parameter of the user is acquired by one or more sensors, and wherein the respiration pattern is adapted in a manner dependent on a degree to which the actual ventilation parameter differs from the setpoint ventilation parameter, such that the specified respiration pattern can be partially adapted to the real respiration pattern of the patient in order to make it easier for the user to synchronize its respiration with the specified respiration pattern.
